(19) United States
(12) Patent Application Publication
(10) Pub. No.: US 2023/0338700 A1
(43) Pub. Date: Oct. 26, 2023

(54) MICROACTUATOR ENABLED SELF-CLEARING SYSTEM AND METHOD

(71) Applicant: Purdue Research Foundation, West Lafayette, IN (US)

(72) Inventors: Hyowon Lee, West Lafayette, IN (US); Qi Yang, San Diego, CA (US)

(21) Appl. No.: 18/136,657

(22) Filed: Apr. 19, 2023

Related U.S. Application Data
(60) Provisional application No. 63/333,253, filed on Apr. 21, 2022.

(51) Int. Cl. A61M 25/00; A61B 8/12

(52) U.S. Cl. CPC A61M 25/0043; A61B 8/12; A61M 2025/0057

(57) ABSTRACT

The self-clearing system includes a microactuator, a catheter lumen, and an actuation device. The microactuator includes a polyimide structural layer and a conduction layer. The polyimide structural layer has a main body and a flexure. The conduction layer is coupled to the polyimide structural layer. The catheter lumen is configured to accept the microactuator. The actuation device is configured to wirelessly engage the microactuator.

### CROSS REFERENCE TO RELATED APPLICATIONS

The present application is a U.S. non-provisional application which claims the benefit of U.S. provisional application Ser. No. 63/333,253, filed Apr. 21, 2022, the content of which is incorporated by reference herein in its entirety.

### GOVERNMENT SUPPORT CLAUSE

This invention was made with government support under NS095287, TR001108, and TR002529 awarded by the National Institutes of Health. The government has certain rights in the invention.

### FIELD

The disclosure generally relates to obstruction clearing systems and, more particularly, to blood clot clearing systems.

### INTRODUCTION

This section provides background information related to the present disclosure which is not necessarily prior art.

Brain hemorrhage is one of the most common and lethal forms of stroke, affecting more than 2 million patients annually worldwide. In 45% of these cases, bleeding occurs inside the ventricles of the brain and leads to intraventricular hemorrhage (IVH). When a blood clot (hematoma) forms and obstructs the circulation of cerebrospinal fluid (CSF), IVH can lead to an even deadlier condition (50-80% mortality) known as post-hemorrhagic hydrocephalus (PHH, 40% of IVH). IVH is especially common in preterm pediatric patients with low birthweight, and subsequent PHH can have devastating neurodevelopmental consequences.

A known treatment of IVH is the rapid removal of hematoma to prevent further deterioration using various interventional methods including open surgery, minimally invasive catheter-based drainage, and thrombolytic agents. These are used to relieve the elevated intracranial pressure (ICP), restore CSF flow, and minimize blood exposure to the ventricles. However, using thrombolytic agents on hemorrhagic patients is controversial due to the elevated risk of additional bleeding. For most IVH patients, external ventricular drainage (EVD), ventricular reservoir devices, or neuroendoscopy are used to remove blood-filled CSF and stabilize increasing ICP.

Unfortunately, there are conflicting reports on the clinical efficacy of drainage devices and there is no established guideline to help determine the proper conditions for EVD implantation. This limitation may partly be due to the difficulty in maintaining the patency of the drainage device in the blood-filled ventricle. To facilitate the blood clot removal, thrombolytic agents are sometimes used with a drainage device for increased clot resolution. However, using fibrinolytic agents on hemorrhagic patients is a highly controversial topic due to the elevated risk of additional bleeding and their unknown long-term effects. The obstruction of drainage devices is a notorious issue recognized for more than half a century. To maintain the patency in drainage systems, catheter flushing often takes place in intensive care units. However, flushing alone is often ineffective in clearing the hematoma, which leads to several replacements of failed drainage devices that also increase the risk of infection and other complications including ventriculitis. The high obstruction rate of these critical medical devices results in significant complications when treating IVH, PHH, and other applications where indwelling catheters are needed. There are efforts to combat the occlusion issues in EVD and other drainage devices. For example, researchers have investigated the use of dual catheters in high volume IVH. There are also reports of using high-intensity focused ultrasound to dissolve clots in situ. However, increasing the number of catheters also increases the risk of infection and the potential concerns of off-target tissue damage remain for the ultrasound-induced clot reduction.

Accordingly, there is a continuing need for an obstruction clearing system that may rapidly remove hematomas while also militating against infection, off-target tissue damage, and other complications including ventriculitis.

### SUMMARY

In concordance with the instant disclosure, a self-clearing system that rapidly removes hematomas while also militating against infection, off-target tissue damage, and other complications including ventriculitis, has surprisingly been discovered. Desirably, the self-clearing system may also be implantable and externally controlled.

The self-clearing system of the present disclosure includes an implantable catheter that may be enabled by microscale magnetic actuators. It should be appreciated that nanoscale actuators are also contemplated. The self-clearing system may be externally controlled so that the magnetic microactuators may rapidly breakdown intraventricular thrombosis with a large-deflection actuation to maintain patency in implantable catheters. In a specific example, the self-clearing system may also be known as smart catheters with magnetic microactuators. Advantageously, the self-clearing system may enhance the reduction of the size of the obstructive hematoma, may improve drainage device reliability, and/or may increase the survival rate of IVH-induced animals. Desirably, the self-clearing system may provide a significant advantage over conventional medical devices because the self-clearing system allows implanted devices to be manipulated in situ without the need for additional surgical intervention using externally applied magnetic fields.

The self-clearing system includes a microactuator, a catheter lumen, and an actuation device. The microactuator may include a polyimide structural layer and a conduction layer. The polyimide structural layer may have a main body and a flexure. The conduction layer may be coupled to the polyimide structural layer. The catheter lumen may be configured to accept the microactuator. The actuation device may be configured to engage the microactuator.

Various ways of manufacturing the microactuator system configured to remove a blood clot are provided. For instance, a first method may include a step of providing a base layer. A release layer may be disposed on the base layer. Then, a polyimide layer may be disposed on the release layer. Afterwards, an etch mask may be disposed on the polyimide layer. The etch mask may be photo-patterned. Then, the polyimide layer may be etched. The etch mask may then be removed. The method may also include a step of disposing a conduction layer on polyimide layer. Then, the conduction layer may be electroplated. Afterwards, the microactuator may be removed from the base layer and the release layer.

Various ways of using the self-clearing system configured to remove a blood clot are provided. For instance, a second method may include a step of providing a microactuator including a polyimide structural layer and a conduction layer. The polyimide structure may have a main body and a flexure. The conduction layer may be coupled to the polyimide structural layer. The second method may further include a step of disposing the microactuator within catheter lumen. Then, the microactuator may be engaged with an actuation device.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a front perspective view of a self-clearing system having a microactuator, a catheter lumen, and an actuation device, further depicting the actuation device engaging the microactuator into a second position, according to one embodiment of the present disclosure;

FIG. 2 is a front perspective view of the self-clearing system, as shown in FIG. 1, further depicting the actuation device not engaging the microactuator, thereby permitting the microactuator to be disposed in a first position, according to one embodiment of the present disclosure;

FIG. 3 is a schematic illustration of one step in a method for manufacturing the self-clearing system, further depicting a polyimide spin coat curing on a single-crystal silicon (Si) wafer and a silicon dioxide (SiO2) release layer, according to one embodiment of the present disclosure;

FIG. 4 is a schematic illustration of another step of the method, as shown in FIG. 3, further depicting the evaporation of an etching mask and defining photoresist for an actuator outline, according to one embodiment of the present disclosure;

FIG. 5 is a schematic illustration of another step of the method, as shown in FIGS. 3-4, further depicting a wet etch of chromium (Cr) and dry etch of polyimide, according to one embodiment of the present disclosure;

FIG. 6 is a schematic illustration of another step of the method, as shown in FIGS. 3-5, further depicting the removal of Cr and evaporation of gold (Au) as a conduction layer, according to one embodiment of the present disclosure;

FIG. 7 is a schematic illustration of another step of the method, as shown in FIGS. 3-6, further depicting the application of photoresist and nickel (Ni) electroplating, according to one embodiment of the present disclosure;

FIG. 8 is a schematic illustration of another step of the method, as shown in FIGS. 3-7, further depicting the removal of photoresist and the remaining Au layer, according to one embodiment of the present disclosure;

FIG. 9 is a schematic representation in a top plan view of a microactuator with a straight beam flexure, according to one embodiment of the present disclosure;

FIG. 10 is a schematic representation in a top plan view of a microactuator with a serpentine flexure, according to one embodiment of the present disclosure;

FIG. 11 is a top perspective view of the self-clearing system having a microactuator disposed within a catheter lumen, further depicting the microactuator disposed in the first position where an actuation device is not engaging the microactuator, according to one embodiment of the present disclosure;

FIG. 12 is a top perspective view of the self-clearing system, as shown in FIG. 11, further depicting the microactuator disposed in the second position where an actuation device is engaging the microactuator, according to one embodiment of the present disclosure;

FIG. 13 is a top perspective view of the self-clearing system having a plurality of microactuators disposed in a catheter lumen, according to one embodiment of the present disclosure;

FIG. 14 is a top perspective view of a microactuator, further depicting a coordinate system (x-z) and a magnetic field (H) angle θ;

FIG. 15 is a line graph illustrating a calculated magnetic torque produced on the ferromagnetic element of a serpentine flexure and a straight flexure under 15 mT at different field angles, according to one embodiment of the present disclosure;

FIG. 16 illustrates a finite element analysis of stress distribution on a serpentine flexure and a straight flexure under 0.1 mN load at a terminal end, according to one embodiment of the present disclosure;

FIG. 17 is a line graph illustrating a maximum calculated deflection under loading condition (0.01-0.1 mN) for a serpentine flexure and a straight flexure from finite element simulation, according to one embodiment of the present disclosure;

FIG. 18 is a line graph illustrating a maximum calculated stress under various deflection conditions for a serpentine flexure and a straight flexure from finite element simulation, according to one embodiment of the present disclosure;

FIG. 19 is a line graph illustrating a static deflection angle prediction and measurements for the serpentine and the straight flexures, further depicting where each data point represents a different experiment using an independent sample, according to one embodiment of the present disclosure;

FIG. 20 is a schematic illustration of the bench-top blood circulation and magnetic actuation setup, according to one embodiment of the present disclosure;

FIG. 21 is a graphical representation of a differential pressure (P2−P1) recording of all control catheters (n=4);

FIG. 22 is a graphical representation of a differential pressure (P2−P1) recording of all flushing catheters (n=6), further depicting frequent pressure spikes associated with each flushing;

FIG. 23 is a graphical representation of a differential pressure (P2−P1) recording of all self-clearing catheters (n=11);

FIG. 24 is a graphical representation of a comparison of time-to-occlusion (TTO) to reach 40 mmHg between the control catheters (n=4), catheters that underwent flushing (n=6), and self-clearing catheters (n=11);

FIG. 25 is a graphical representation of a comparison of the total time over the threshold (TOT) between the control catheters (n=4), catheters that underwent flushing (n=6), and self-clearing catheters (n=11);

FIG. 26 is a schematic diagram illustrating a timeline for the experimental study which includes implantation of the self-clearing system, a computer tomography scan, and actuating the self-clearing system;

FIG. 27 is a graphical representation illustrating a box plot of ventricle volume until week 1 where the box plot shows the interquartile range and the horizontal lines within the boxes are median, further depicting the control animals with traditional catheters had a significant increase in ventricle volume by week 1;

FIG. 28 is a graphical representation illustrating a Kaplan-Meier survival plot with corresponding risk table, further depicting that, by week 1, the traditional shunt systems in all control animals had failed whereas 80% of the shunt systems with self-clearing catheters remained obstruction-free with biweekly actuation;

FIG. 29 is a flow chart of a first method for manufacturing a self-clearing system, according to one embodiment of the present disclosure; and

FIG. 30 is a flow chart of a second method for using a self-clearing system, according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the FIG. is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

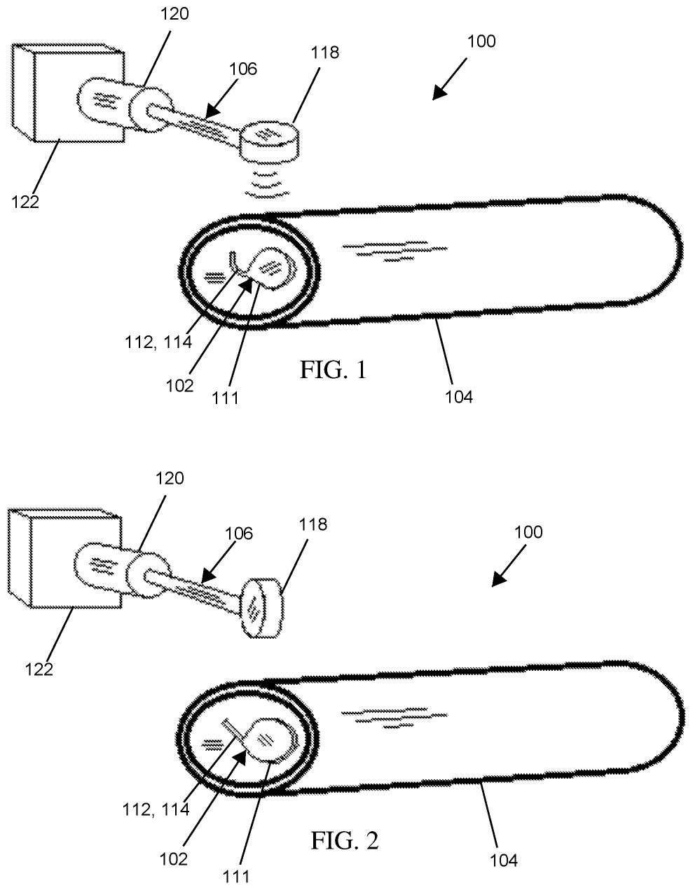

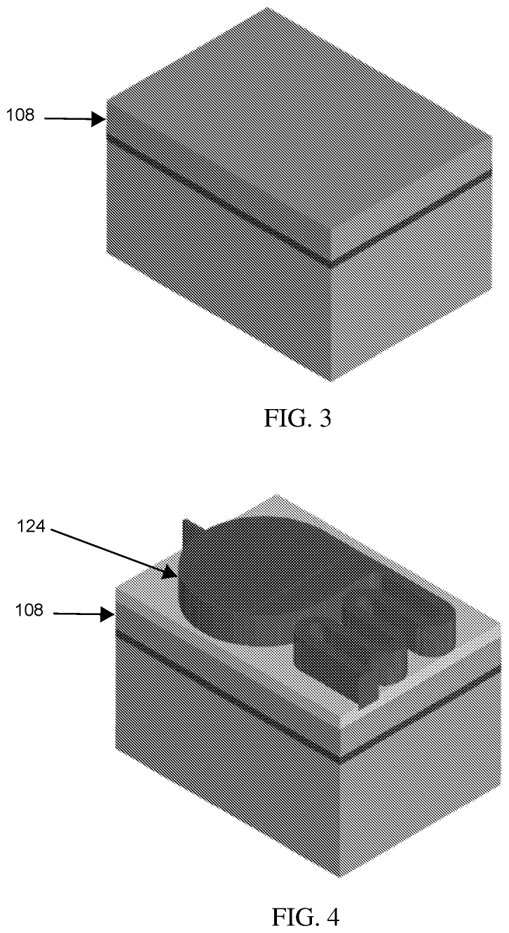

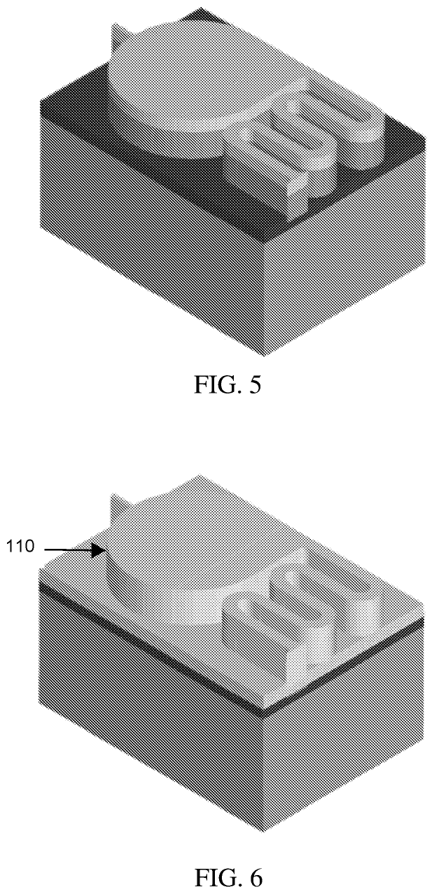

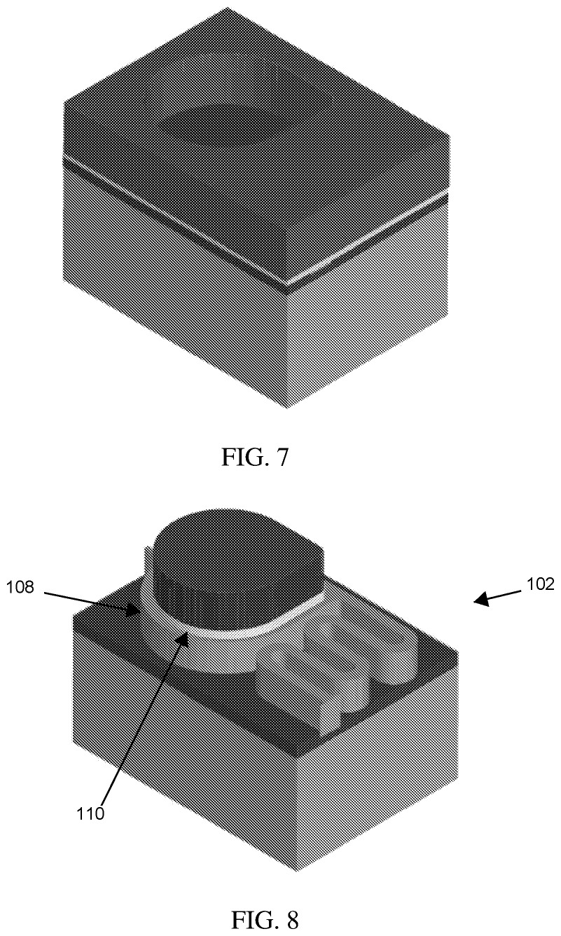

As shown in FIGS. 1-2, the self-clearing system 100 includes a microactuator 102, a catheter lumen 104, and an actuation device 106. With continued reference to FIGS. 3-8, the microactuator 102 may include a polyimide structural layer 108 and a conduction layer 110. The microactuator 102 may include a main body 111 and a flexure 112, 114 formed by the polyimide structural layer 108. The flexure 112, 114 coupled to the main body 111 at a first terminal end 116 of the flexure 112, 114. The conduction layer 110 may be coupled to the polyimide structural layer 108. More specifically, the conduction layer 110 may be electroplated to the polyimide structural layer 108. The catheter lumen 104 may be configured to accept the microactuator 102. The actuation device 106 may be configured to wirelessly engage the microactuator 102.

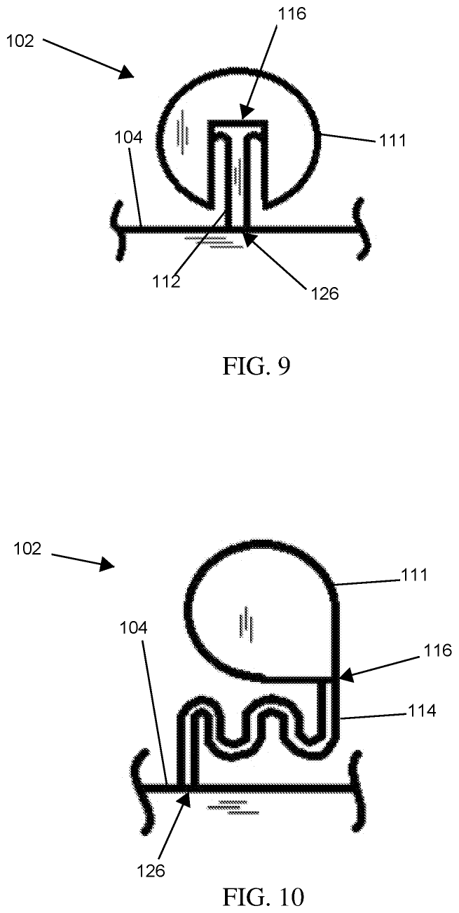

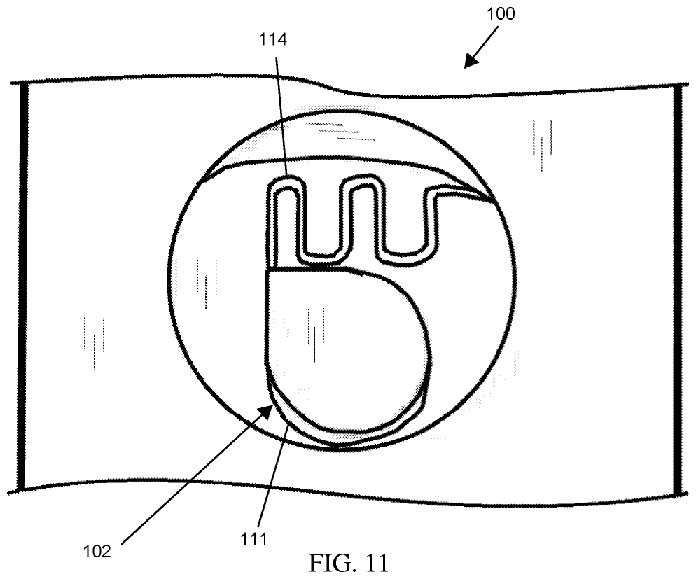

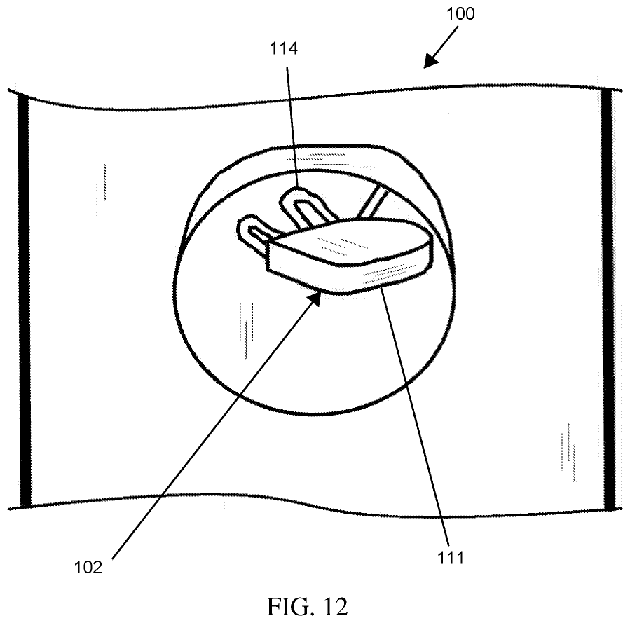

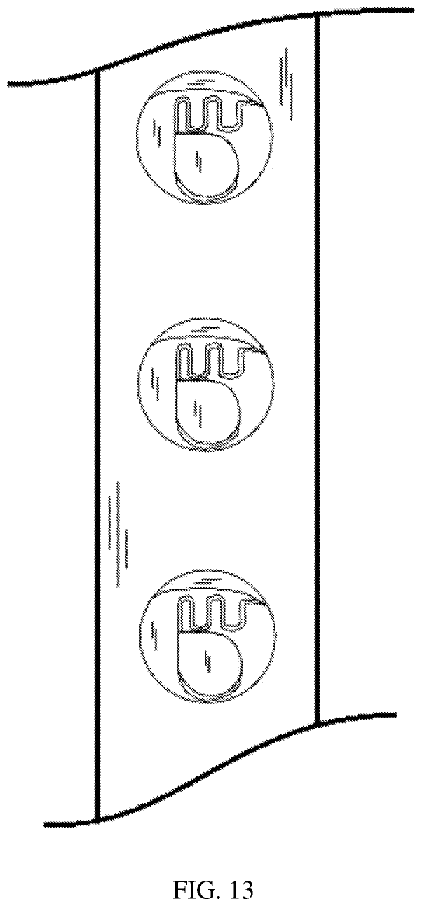

In certain circumstances, the microactuator 102 may include a plurality of functions and may be manufactured from various materials. For instance, as shown in FIG. 3, the polyimide structural layer 108 of the microactuator 102 may include a layer of polyimide PI-2525 commercially available from HD Microsystems, LLC. The flexure 112, 114 may be configured to couple the main body 111 of the microactuator 102 to the catheter lumen 104. The flexure 112, 114 may substantially pliable. In other words, the flexure 112, 114 may bend, twist, and/or flex. As shown in FIGS. 11-12, the substantially pliable flexure 112, 114 may permit the main body 111 to move, rotate, and/or pivot within the catheter lumen 104. As shown in FIG. 9, the flexure 112, 114 be a straight beam flexure 112 having a rectangular-cross sectional shape and provided as a cantilever. It should be appreciated that various geometries may be utilized to form the polyimide structural layer 108. The straight beam flexure 112 shaped as a rectangular cantilever may include a substantially straight polyimide structure coupling the main body 111 of the polyimide structural layer 108 to the catheter lumen 104. Advantageously, the straight beam flexure 112 may have enhanced mechanical robustness. Alternatively, as shown in FIG. 10-12, the flexure 112, 114 may be provided as a serpentine flexure 114 having a curved cross-sectional shape. In a more specific example, the serpentine flexure 114 may have a serpentine cross-sectional shape. In other words, the serpentine flexure 114 may have a plurality of curves. In a more specific example, the serpentine flexure 114 may resemble a switch-back design. The serpentine flexure 114 may couple the main body 111 of the polyimide structural layer 108 to the catheter lumen 104. The conduction layer 110 of the microactuator 102 may include a gold material. The conduction layer 110 may be coupled to the polyimide structural layer 108 by electroplating with a solution that includes at least one of nickel sulfamate, boric acid, and/or sodium dodecyl sulfate. In another specific example, the self-clearing system 100 may include a plurality of microactuators 102 disposed within a single catheter lumen 104, as shown in FIG. 13.

In certain circumstances, the actuation device 106 may include a plurality of functions and may be manufactured from various materials. For instance, the actuation device 106 may include a magnet 118 configured to interact with the microactuator 102. In a specific example, the magnet 118 may be selectively engaged to periodically provide varying interactions with the microactuator 102. In a more specific example, the magnet 118 may be rotated in a manner that intermittently provides a magnetic attraction to the microactuator 102. In an even more specific example, the magnet 118 may be rotated by a motor 120. In a particular example, the actuation device 106 may further include a controller 122 coupled to the motor 120. The controller 122 may engage the motor 120 to rotate the magnet 118 at a predetermined speed. The controller 122 may also engage the motor 120 to rotate the magnet 118 at a variable speed, where a user may select the rate of interactions and/or the duration of each interaction between the microactuator 102 and the magnet 118. For instance, the rate of interactions may include rotating the magnet 118 to engage the microactuator 102 a certain number of times per minute. The duration of each interaction may include rotating the magnet 118 at a speed which controls how much time each engagement of the microactuator 102 takes. One skilled in the art may select other suitable ways of wireless engaging the microactuator 102, within the scope of the present disclosure.

Various ways of manufacturing the self-clearing system 100 configured to remove a blood clot are provided. In certain circumstances, the microactuator 102 may be manufactured using surface micromachining techniques, as shown in FIGS. 3-8. FIGS. 3-8 are provided merely to represent the different layers of one embodiment of the self-clearing system 100. Accordingly, FIGS. 3-8 may not be drawn to scale. More particularly, the different layers may include different thicknesses than what is illustrated in FIGS. 3-8. For instance, a first method 200 may include a step 202 of providing a base layer. As a non-limiting example, the base layer may include a silicon wafer. A release layer may be disposed on the base layer. In a specific example, the release layer may include around 50 nm of silicon dioxide deposited on the base layer by plasma-enhanced chemical vapor deposition. Then, a polyimide structural layer 108 may be disposed on the release layer. More particularly, the polyimide structural layer 108 may include a layer of polyimide PI-2525 spun coated at around 1750 rpm and cured in a nitrogen oven up to around 350 degrees Celsius. The thickness of the polyimide structural layer 108 may be up to around 11 μm. The polyimide structural layer 108 may provide a main body 111 and a flexure 112, 114 of the microactuator 102. Afterwards, an etch mask 124 may be disposed on the polyimide structural layer 108. In a specific example, the etch mask 124 may include a layer of chromium that is around 50 nm thick. In a particular example, the etch mask 124 may be evaporated on the polyimide structural layer 108. The etch mask 124 may be photo-patterned using a microchemical such as AZ9260. Then, the polyimide structural layer 108 may be etched with a chromium etchant such as a Cr-16 that is commercially available from KMG Chemicals. The etch mask 124 may then be removed. The conduction layer 110 may include around 100 nm of gold. Then, the conduction layer 110 may be coupled to the polyimide structural layer 108, for instance, by electroplating the conduction layer 110. In a specific example, the electroplating may use a 200 μm thick electroplating mold that is photo-patterned using a negative photoresist. Afterwards, the microactuator 102 may be removed from the base layer and the release layer. In a particular example, the microactuator 102 may be released from the base layer using a buffered oxide etchant. In a specific example, the microactuator 102 may be rolled and inserted into a catheter lumen 104. A skilled artisan may utilize other methods and/or materials for manufacturing the microactuator 102, within the scope of the present disclosure. In certain circumstances, a second terminal end 126 of the flexure 112, 114 may be coupled to a catheter lumen 104. In a specific example, the second terminal end 126 may be coupled to an interior wall of the catheter lumen 104. Next, an actuation device 106 may be disposed separate from, but adjacent to the microactuator 102, so that the actuation device 106 may selectively and intermittently engage the microactuator 102 wirelessly.

Various ways of using the self-clearing system 100 configured to remove a blood clot are provided. For instance, a second method 300 may include a step 302 of providing a microactuator 102 including a polyimide structural layer 108 and a conduction layer 110. The polyimide structure may have a main body 111 and a flexure 112, 114. The conduction layer 110 may be coupled to the polyimide structural layer 108. The second method 300 may further include a step 304 of disposing the microactuator 102 within catheter lumen 104. The microactuator 102 may be coupled to the catheter lumen 104. Then, the microactuator 102 may be engaged with an actuation device 106. In a specific example, the actuation device 106 may include a magnet 118 which wirelessly engages the microactuator 102 with a magnetic force. In a more specific example, the actuation device 106 may further include a motor 120 which rotates the magnet 118 to intermittently engage the microactuator 102. In an even more specific example, the actuation device 106 may further include a controller 122 which adjusts at least one of a rate and a duration of the engagement between the magnet 118 and the microactuator 102. One skilled in the art may select other suitable methods for using the self-clearing system, within the scope of the present disclosure.

Using the self-clearing system 100 as an implantable magnetic microactuator 102, the self-clearing system 100 can advantageously generate large enough forces to break down obstructive blood clots by applying time-varying magnetic fields. Desirably, in a blood-circulating model, the self-clearing system 100 demonstrated a >7× longer functionality than traditional catheters (211 vs. 27 min) and maintained a low pressure for longer periods (239 vs. 79 min). Using a porcine IVH model, the self-clearing system 100 exhibited an enhanced survival rate in comparison to control catheters (86% vs. 0%) over the course of 6 weeks. Treated animals also had significantly smaller ventricle sizes one week after implantation compared to the control animals with traditional catheters. The self-clearing system 100 provided as a magnetic microactuator-embedded smart catheter may expedite the removal of blood from the ventricles and militate against IVH.

### III. EXAMPLE

Provided as a specific, non-limiting example, one embodiment of the self-clearing system 100 was produced by providing a serpentine flexure 114 with tear-drop-shaped ferromagnetic elements. The serpentine flexure 114 may consist of four windings with five 400 μm-long straight segments connected by four arcs providing 100 μm gaps in between them. The serpentine flexure 114 provides a six-fold smaller bending stiffness and enhanced deflection within a limited footprint. In addition to improved beam design, the large aspect ratio magnetic elements provide additional magnetic torque compared to the previous versions despite having nearly identical volume because of magnetic geometry anisotropy. The microfabricated thin film microactuators 102 were then integrated into a custom catheter lumen 104 to create a self-clearing ventricular catheter 100. FIGS. 11-12 illustrate a typical actuation motion of a microactuator 102 with serpentine flexure 114. In the presence of time and spatially varying magnetic fields, the actuator deflects in and out of the plane for a more dynamic hematoma removal. Dynamically, the actuator featured the largest actuation amplitude near 10-20 Hz in water.

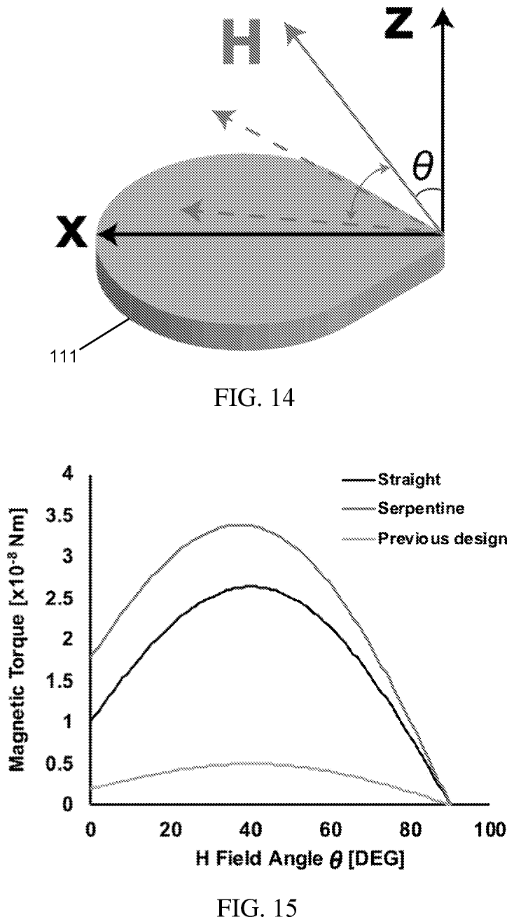

FIGS. 14-19 demonstrates some advantages of the magnetic microactuator 102 with the serpentine flexure 114 over the straight beam design flexure 112. The magnetic properties of the electroplated ferromagnetic elements were characterized using a magnetometer to calculate the magnetic torque assuming a field-dependent, non-saturated magnetization. FIGS. 14-15 show that the magnetic torque varies as the function of applied magnetic field angle θ and that maximum torque can be achieved at 40°. As much as a 75% improvement was identified when θ=40°.

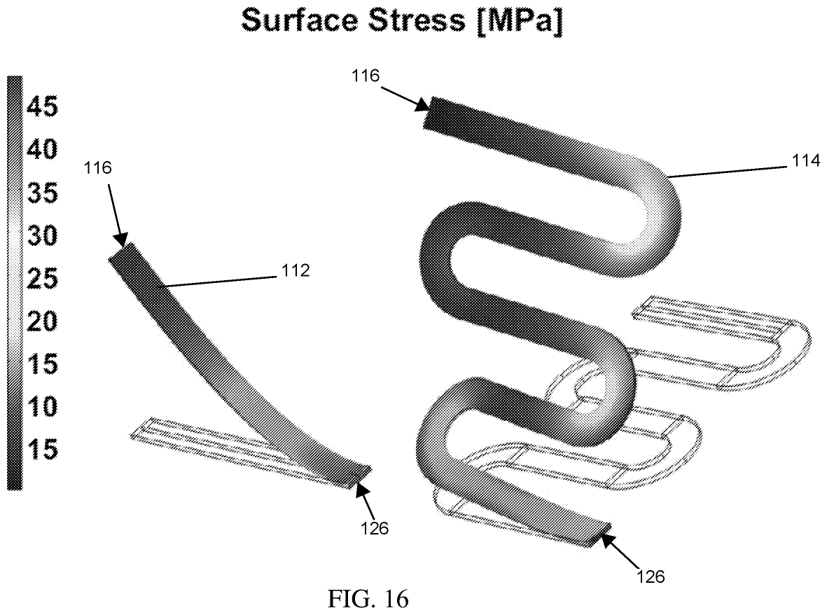

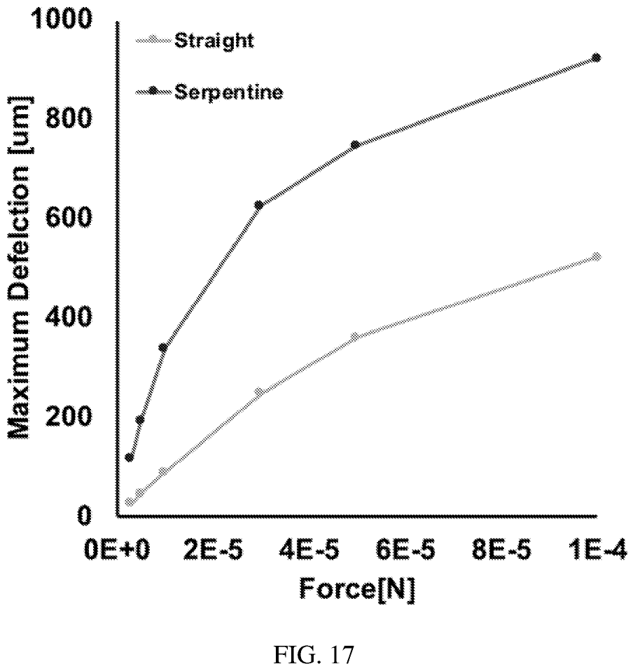

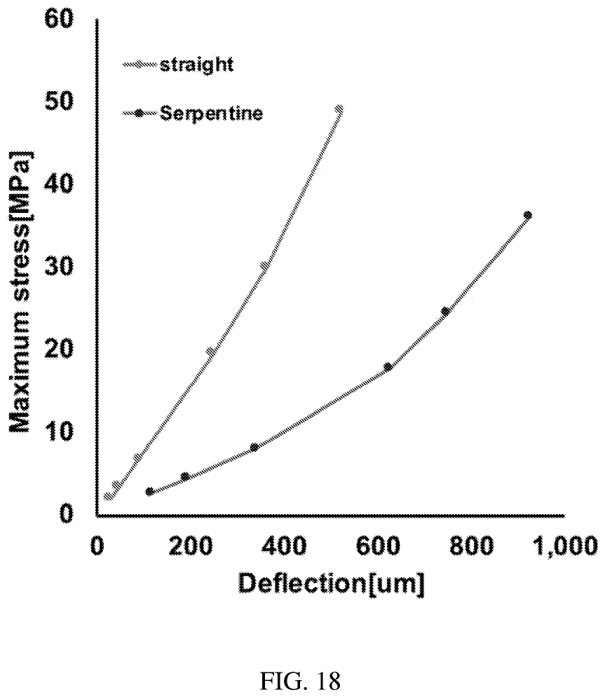

The serpentine flexure 114 design improved actuator displacement per applied magnetic field strength. The numerical analysis showed a larger deflection and smaller stress on the serpentine flexure 114 than the straight beam flexure 112 when a vertical point load was applied to its tip, as shown in FIG. 16. FIGS. 17 and 18 shows varying degrees of deflection and corresponding maximum stress on the beam at various load ranges. The serpentine flexure 114 provided twice as much displacement over the straight beam flexure 112 over the same loading condition. The maximum static stress calculated for serpentine flexure 114 was 36 MPa at 0.1 mN, which is significantly lower than the tensile stress of this polyimide (131 MPa).

The 0.1 mN loading was chosen from the MR safety perspective. A typical human whole-body MR system produces a maximum spatial gradient between 10 and 50 mT/m. Assuming the ferromagnetic element was fully saturated, the 0.1 mN load corresponds to the magnetic force produced from a gradient of 4 T/m, an order of magnitude greater than the physical constraint. Therefore, it is believed to be unlikely that the self-clearing system 100 will be damaged due to mechanical deformation in an MR system.

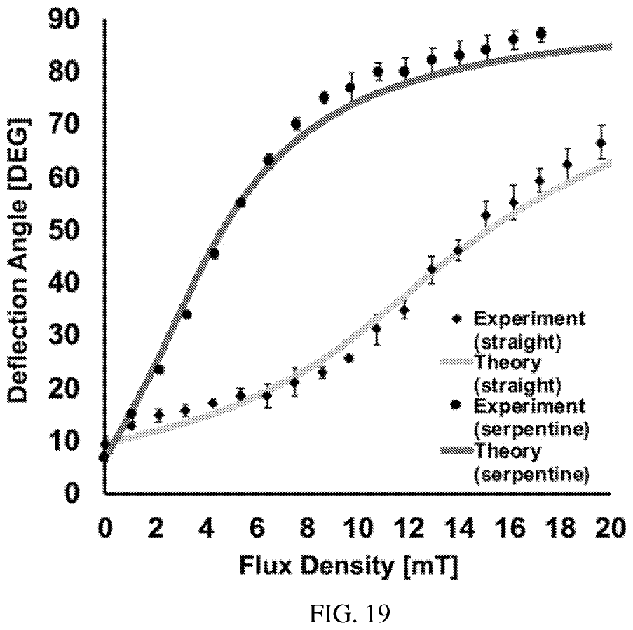

The improvements in magnetic torque and mechanical compliance of the serpentine flexure 114 allow the microactuator 102 to achieve a greater deflection per given magnetic flux density, as show in FIG. 19. Although the microactuators 102 with a straight beam design were effective against protein and cellular biofouling, those were not as effective against macroscopic hematoma. By increasing the magnetic torque and rapid displacement, as shown in the present disclosure, a more robust thrombotic mass that plagues drainage devices in IVH patients could be removed more effectively. For instance, the microactuator 102 with the serpentine flexure 114 achieved >80° deflection with 15 mT compared to only 30° for the straight beam design.

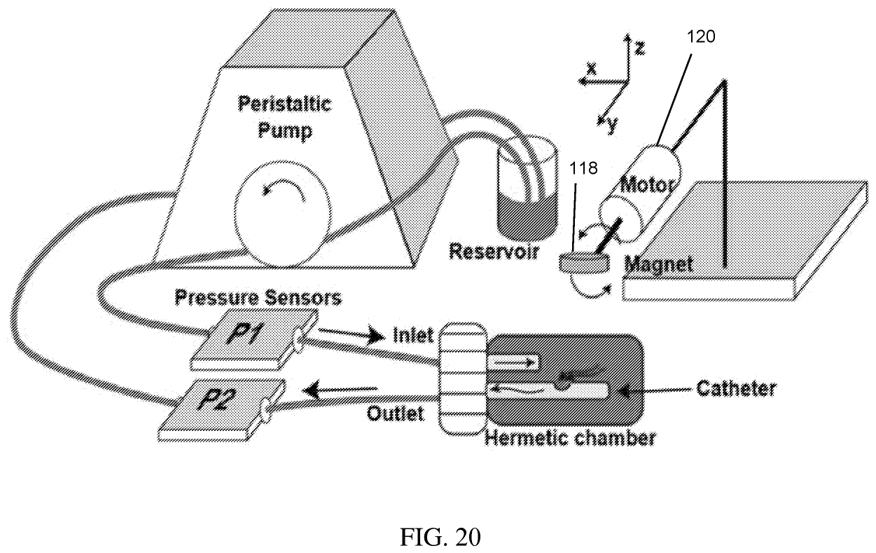

To demonstrate the blood-clot removal capabilities of the self-clearing system, an in vitro circulation system was utilized. Although several groups have reported a reduction of hematoma mass using a static condition, none have demonstrated an effective capability to militate against clots in a continuous flow environment, which is more physiologically relevant for one specific target application. FIG. 20 shows the experimental setup, which mimics a fixed-volume ventricle that circulates sanguineous phosphate-buffered solution (PBS) as a CSF substitute. Using a peristaltic pump, diluted porcine blood (50:50 with PBS) was pumped out of the sealed chamber through different catheter designs. The blood-PBS mixture ratio was determined experimentally to reliably produce hematoma within four hours. As the fluid flowed through the circulation system, a time-varying magnetic field was applied on all three groups: control, flushing, and self-clearing catheters 100. The flushed catheters were identical to control catheters but had a three-way stopcock valve attached to the line of the drainage device with one connector having a syringe with 2 mL of PBS. Once five minutes of pressure above the established threshold (40 mmHg) was reached, the valve was redirected to allow flow from the syringe, and the saline injection was introduced. After the full saline injection, flow in the circulation system was re-established. Flushing was repeated as many times as necessary in the four-hour experiment.

In general, the self-clearing system 100 with an integrated microactuator 102 exhibited a smaller hematoma mass over their inlet pores compared to the flushing and control catheters. The effect of magnetic microactuation on the hematoma was identified by analyzing the structure using a scanning electron microscope (SEM). Utilizing a grading scale developed and analyzed by a board-certified clinical pathologist, the fibrin mesh network significantly differed between the control and treatment devices (p<0.001). The fragments from the treatment device displayed almost no fragments of fibrin, further confirming that the self-clearing system 100 can advantageously break apart hematoma to allow drainage through the ventricular catheter.

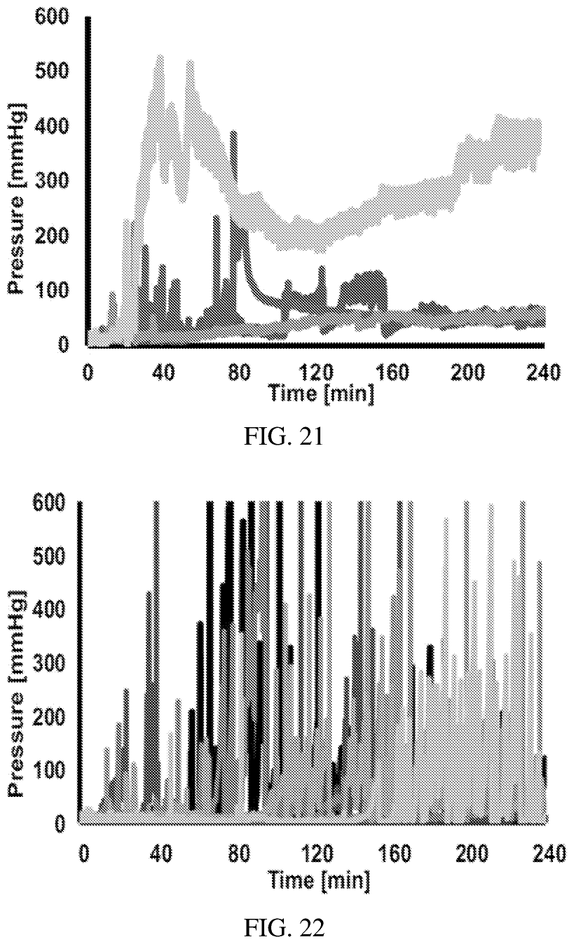

The impact of magnetic microactuation was quantified by measuring the differential pressure between the inlet and the outlet during the experiment. FIGS. 21-23 show differential pressure throughout the experiments. Without any blood in circulation, the differential pressure ranged between 5 and 15 mmHg, which is comparable to the normal intraventricular pressure of a patient. However, with blood, the control catheters exhibited an exceptionally high average pressure of 100±111 mmHg, and the flushing catheters had an average pressure of 41±10 mmHg, whereas the self-clearing catheters 100 had an average pressure of 11±31 mmHg. To compare all catheter groups, a threshold pressure of 40 mmHg was chosen because ICP exceeding this value is considered life-threatening. The time-to occlusion (TTO) was defined by the time to reach this threshold. TTO was postulated to indicate how quickly the drainage system shows functional deterioration due to hematoma. For control catheters, the average TTO was 27±18 min. For flushing catheters, the average TTO was 29±18 min, which is similar to the control catheters. This was expected as both devices are single-pore catheters without the actuator. For the treatment group with the self-clearing catheter 100, the average TTO was delayed to 104±86 min for straight flexure devices 104 and 211±72 min for serpentine flexure devices 104.

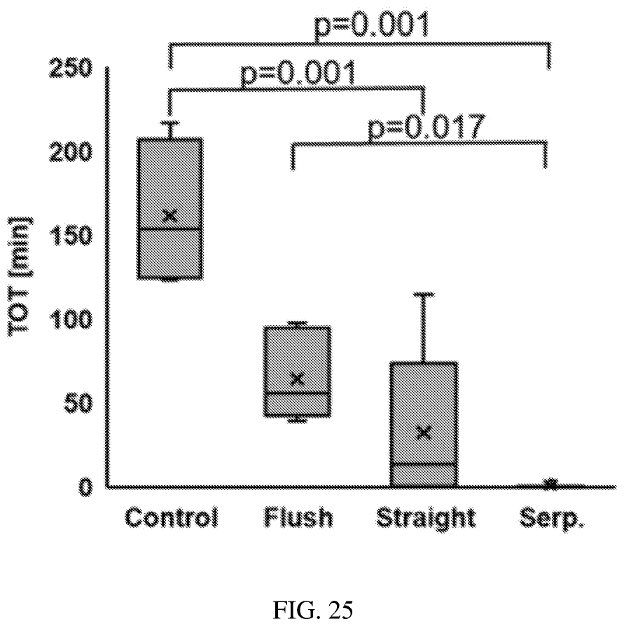

The total time over the threshold pressure of 40 mmHg (TOT) was also determined. The TOT may indicate the resilience of the drainage system to combat blood-clot-induced failure. The average TOT for actuation devices 106 was 162±44 min during a 240 min experiment. The average TOT for the flushing catheters was 64±25. In comparison, the average TOT was 33±48 min for the catheters with straight devices and 0.3±0.8 min for the catheters with serpentine devices. FIGS. 24 and 25 compare the TTO and TOT for each condition. Even at a lower threshold pressure (20 mmHg), the self-clearing system 100 still exhibited a significantly better performance in terms of the TTO and TOT than the control and the flushed catheters.

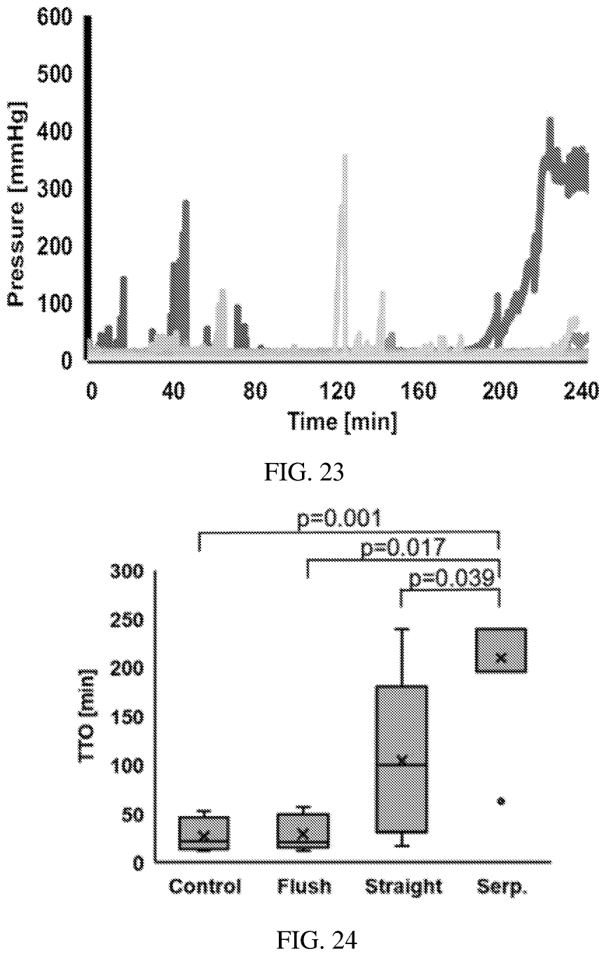

In all four control catheters, the differential pressure remained above the threshold at the end of the circulation period, indicating robust and sustained obstruction by a hematoma, as shown in FIG. 21. Three out of six catheters used for the flushing test had remained above the pressure threshold, indicating large hematoma in the system was still present and was not absolved by the flushing. The average number of flushes per experiment run (n=6) was 8±3 resulting in high-pressure spikes outside of the pressure range of interest throughout the experiment time. Conversely, 8 out of 11 self-clearing system catheters 100 (5 straight and 6 serpentine devices) exhibited relatively lower pressure (<20 mmHg) at the end of the circulation, as shown in FIG. 23. Compared to the control catheters, less frequent pressure spikes in the self-clearing system 100 were also observed.

These results demonstrate that the self-clearing system 100 may significantly delay occlusion due to hematoma and improve device reliability. The experiments also show the cumbersome nature of having an experienced clinician be vigilant for obstruction and applying timely flushing with a risk of pressure spikes. In comparison, the self-clearing system 100 can easily be actuated using external control with minimal risk for pressure spikes. Moreover, these results confirmed that the larger actuation deflection afforded by the serpentine flexure 114 design could improve blood-clot removal capabilities. Without being bound to any particular theory, it is believed the rapid translational motion of the microactuator 102 in the self-clearing system 100 causes localized shear at the catheter inlet, which exceeds the threshold above which hematoma becomes incapable to attach and accumulate on the catheter surface. It is also contemplated to use numerical evaluation to estimate the amount of shear stress the microactuator 102 in the self-clearing system 100 generates in a circulating flow environment.

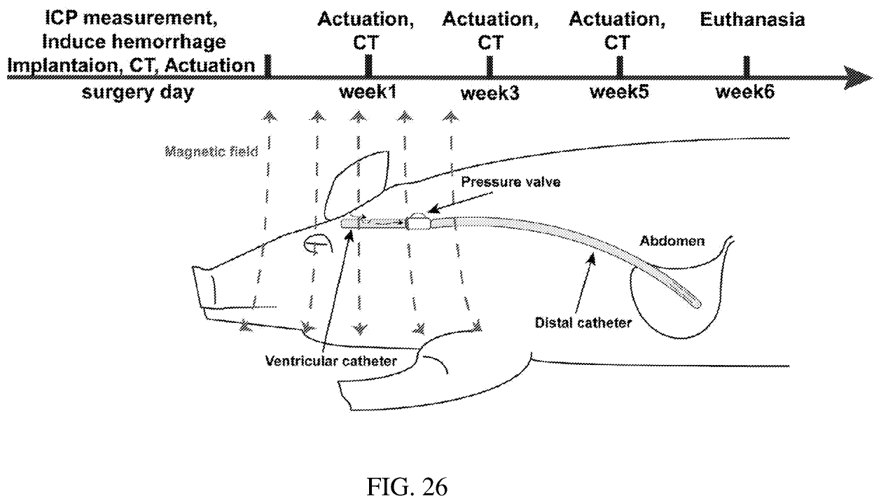

To verify the results from our in vitro experiment, a porcine model of IVH was developed and evaluated the impact of self-clearing catheters 100 in vivo. Initially, preliminary studies were performed with six pigs to determine the amount of blood to be injected to cause large hematoma in the ventricle and sustained intracranial hypertension. As a result of these studies, 10 ml of blood admixed with 140 units of thrombin immediately prior to injection was identified as a reliable way to cause IVH and subsequent PHH. The blood and thrombin were injected into the right lateral ventricle in three equal aliquots. The timeline of the in vivo evaluation can be seen in FIG. 26.

A total of thirteen pigs were used for the evaluation. The median weight was 28.0±3.9 kg. The baseline ICP was 11±5.2 mmHg. As the blood-thrombin injection was paused whenever ICP was ≥50 mmHg, the injection was performed over 2 to 15 min (median, 8 min). During injection of the third and final aliquot of blood, ICP would typically immediately increase above 50 mmHg with each injection of about 1 ml, then quickly fall. The ventricular catheter was placed 7.5±7.0 min after the blood-thrombin injection was completed. A custom ventricular catheter with a single inlet pore was again used. ICPs reached a high but variable peak, fell quickly initially, then slowly declined to reach a plateau of around 13±4 mmHg. The custom ventricular catheter was connected to a conventional one-way valve and peritoneal catheter to form a ventriculoperitoneal (VP) shunt. The fully implanted system was used in place of conventional EVD to mitigate the potential risk of surgical site infection and to allow free movement for the recovered animals over the 6-week experimental duration.

In all pigs, postoperative computed tomography (CT) was utilized to confirm the accurate intraventricular injection of blood and air. The post-shunting CT also confirmed the correct location of the ventricular catheter. In one Control and one Treatment pig, it was determined that the only inlet pore had passed through the lateral ventricle and into the brain parenchyma. In both of these pigs, a brief second surgery was performed to retract the ventricular catheter and the subsequent CT confirmed the correct ventricular placement of the inlet pore.

All six Control pigs suffered sudden neurological decline and were found to have shunts obstructed with hematoma at necropsy. The sudden neurological decline in the Control pigs occurred twelve hours to five days postoperatively (median, three days). Four of these animals became moribund within hours of the first sign of deterioration and were euthanized. Post-mortem CT revealed marked enlargement of the ventricles since the post-operative CT, transtentorial with or without cerebellar herniation, and continued correct ventricular location of the ventricular catheter without shunt displacement. The other two control pigs showed a sudden, severe but non-fatal neurological decline. The ventricles were markedly larger on the next CT, and for the rest of the study. All explanted VP shunts in the control group showed evidence of hematoma inside their lumen. There was a total of eight hematomas in these six VP shunts (three ventricular catheters, three valvular, two distal catheters).

On the contrary, only one of the seven Treatment pigs suffered neurological decline and was found to have a complete shunt obstruction with a hematoma at necropsy. This animal deteriorated twelve hours post-operatively, rapidly became moribund, and was euthanized. This was the pig that had had a second surgery to slightly retract the ventricular catheter, resulting in delayed actuation. Upon post-mortem examination, a further enlargement of the ventricles and persistence of intraventricular hematoma was discovered. Hematoma blocking the push connector (inlet for the valve) was found at necropsy. The valve itself was filled with sanguineous CSF but was not obstructed. This was the only fatal hematoma in the seven VP shunts of the Treatment pigs. The ventricular catheters, valves, and distal catheters were otherwise patent without any evidence of obstructive hematoma. These observations suggest that magnetic actuation may be able to preserve the patency of the downstream drainage path despite being at the proximal end of the ventricular catheter.

One Control pig suffered from non-fatal shunt obstruction with a hematoma on day four, followed by dehiscence of the wound edges, post-operative infection, and euthanasia. Two Treatment pigs developed a fatal infection. One was euthanized on day seven for peri-operative infection. The other rubbed his head on cage bars post-operatively. The skin edges were dehisced and were surgically re-closed on day five and again on day seven. Twelve days post-operatively his appetite decreased and on day thirteen post-operatively he was severely depressed, displayed nystagmus, and was euthanized.

In all three infected pigs, ventriculomegaly was seen at post-mortem CT, and during necropsy, purulent discharge was observed centered around the valve and surgical site and tracking down the ventricular catheter into the brain. In the two Treatment pigs, the purulent discharge obstructed the inlet pore. In the Control pig, there was also a hematoma obstructing the lumen of the ventricular catheter. After two infections in the first seven surgeries, two peri-operative doses of florfenicol were administered to each animal and added a subcutaneous muscle suture layer.

The postoperative CT in the animals showed significantly larger ventricles than prior to surgery in both groups (p=0.002). By week 1, all air had been resorbed. In the Treatment group, all hematomas had been resorbed and ventricular size had non-significantly decreased. In the Control group, there had been a further enlargement of the ventricles and in some animals, hematoma persisted.

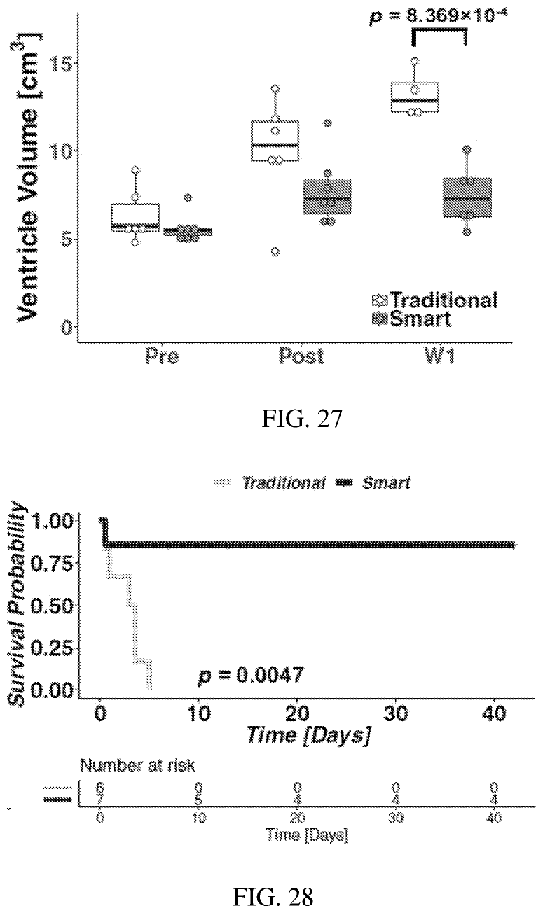

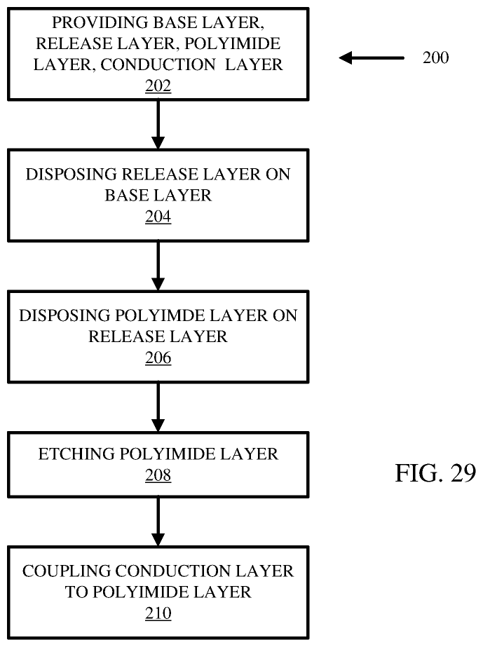

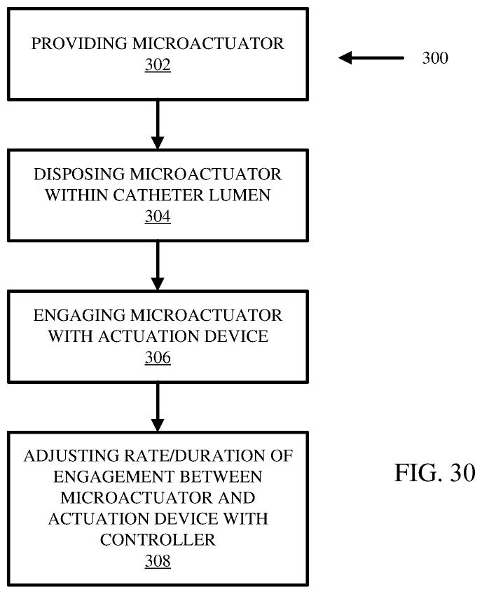

FIG. 27 shows the change in ventricular volume until 1-week post-implantation. Two-way ANOVA showed statistical significance for both the device type and time. Specifically, the ventricular volume in the Control animals was significantly larger than in the Treatment group (p<0.001). Moreover, the ventricular volume significantly increased before and after IVH and after a week of recovery (p<0.001). Pairwise comparison showed that by week one, there was a significant difference in ventricular volume between the Control and the Treatment group (p<0.001).

The data from week 3 and week 5 were omitted for statistical comparison because all Control shunts failed after W1. Although the Treatment animals survived longer with the self-clearing system, the ventricle volume continued to increase. It is contemplated that the self-clearing system 100 is prolonging the functional lifetime, and it may be advantageous to increase the actuation duty cycle to optimize treatment.

A Kaplan-Meier analysis was performed to evaluate the survivability of the shunt system due to hematoma, censoring the death of pigs due to infection, as shown in FIG. 28. The median shunt survival in the Treatment group was forty-two days and the median shunt survival in the Control group was three days. This difference was statistically significant (p=0.0047), which further supports that the self-clearing system 100 is more reliable and capable than existing drainage devices. The Kaplan-Meier analysis was repeated considering infection as a shunt failure, but the difference still remained statistically significant and the p-value unchanged (p=0.0047), which further supports that the self-clearing system 100 may improve the outcomes of IVH and PHH patients.

For the treatment of IVH, rapid removal of hematoma is critical in preventing further injury to the brain. Although EVDs may be used to facilitate hematoma clearance, it is recognized that VP shunts are not used for immediate treatment of IVH. To demonstrate the long-term efficacy of the self-clearing system 100 in vivo, however, it was necessary to use the self-clearing system 100 as a part of a fully implanted shunt system since it would have been nearly impossible to maintain the sterility of the transcutaneous EVD in the porcine model of the present disclosure over six weeks.

It is also important to note that the tested prototype catheters have a much higher risk of failure than a standard ventricular catheter. Conventional ventricular catheters typically contain 16 or more inlet pores (3 to 4 rows of 4 to 8 inlet pores). The experimental, non-limiting self-clearing system 100 had a single inlet pore to accelerate the occlusion. As such, the occlusion of this lone inlet pore would have led to an accelerated and complete device failure. Despite not having redundant pores for secondary flow, the results of the present disclosure show that the single-pore device can maintain its patency due to periodic magnetic actuation. It should be appreciated that an array of inlet pores may be integrated into the catheter.

The single inlet pore also increased the chance of inadequate surgical placement: the rows of inlet pores in conventional catheters maximize the likelihood that multiple inlet pores will be located in the lateral ventricle. In one Treatment pig, the post-operative CT indicated that this single inlet pore was too deep (passing through the ventricle and into brain tissue on the other side) and led to additional surgery and, most importantly, delayed actuation. This Treatment shunt was not actuated until after a second surgery to retract the ventricular catheter and was fatally obstructed in less than twenty-four hours. Upon explantation, it was found that the hematoma had passed the microactuator 102 within the tip of the ventricular catheter and was at the junction of the ventricular catheter and valve, suggesting the first actuation may not have occurred until after overwhelming hemorrhage had already passed the actuator. All other Treatment shunts were first actuated soon after placement and subsequently remained free of hematoma obstruction throughout the six-week study.

There was an additional risk of device failure as the catheter was placed directly into the ventricular hematoma, through the tract used for injection of blood, being inserted immediately after the injection was completed. Since the blood was mixed with the coagulant agent thrombin, we suspect that a mixture of CSF, blood, and thrombin immediately entered the catheter to begin coagulating. In current clinical practice, the presence of hematoma is a contraindication for shunting, which is typically recommended only when hydrocephalus has been diagnosed. This extended waiting period can further exacerbate the brain injury due to the mass effect and result in a poor clinical outcome. Thus, the present disclosure may represent a significant improvement over the state-of-the-art in IVH treatment. Despite the added risk of device failure, the robustness of the approach outlined in the present disclosure enables earlier usage of drainage systems using the self-clearing system.

The effect of having a valve as a part of the VP shunt should also be carefully examined. Conventional EVDs typically do not have an implanted valve, which is often used to prevent over-drainage of CSF and siphoning effect for patients with VP shunts. The presence of an implanted valve with small fittings that connects to the proximal catheter likely has exacerbated the shunt obstruction. However, the experimental results indicate that the actuated self-clearing catheter 100 may also be able to prolong the lifetime of downstream components (i.e., pressure valve) as well, potentially by reducing the size of the blood clot that passes through the drainage pathway.

A commonly used valve in VP shunt surgery has a medium opening pressure (6-7 mmHg), which is within the range for normal ventricular pressure of 5-15 mmHg. Without being bound to any particular theory, it is believed that the low opening pressure may have increased the passage of hemorrhagic CSF through the VP shunt, as the coagulating fluid would have taken the path of least resistance rather than negotiated the ventricular system. Hemorrhage present within the ventricular system would be able to form a semi-permanent and permanent obstructive thrombus, rather than being dislodged by CSF flow. This would mandate the shunt system to remain functional to keep the patient alive, which further highlights the potential utility of the approach described in the present disclosure to maintain the patency of a drainage system using a self-clearing catheter 100. Although the experimental design of the present disclosure did not explore the mechanism of clinical deterioration, it is contemplated to characterize the change in CSF perfusion using a periodic CT perfusion or laser Doppler flow.

It is also important to note that the injection of blood in the in vivo experiment provides a substrate for bacterial growth. Typically, up to 20% of shunts fail due to infection-related issues. This is exacerbated in neonates with PHH for whom 71% of shunt failures can be attributed to infection. It is contemplated to use a combination of microactuators 102 with antibiotic or anti-inflammatory drug-eluting catheters to ensure that the self-clearing system 100 can not only accelerate hematoma clearance, but it may provide robust protection against infection-related device failures.

The performance of the self-clearing system 100 against the common clinical practice of using saline flush to remove drainage device obstruction was also compared. Although saline flushes are effective in removing the obstruction, each flushing caused a potentially dangerous level of a pressure spike, and the catheter was rapidly re-occluded, which necessitated more frequent flushing. Pressure spikes notwithstanding, the flushed catheters remained above the safe pressure range for longer periods than the self-clearing system. Our results highlight the clinical challenge of having a caretaker constantly monitoring patient ICP to ensure that the drainage device performs reliably until the hematoma clears. In comparison, the approach of the present disclosure may be used with minimal user input using an overhead electromagnet that can be placed over the patient's head. Furthermore, by optimizing the actuation duty cycle, it may be possible to improve the performance and utility of drainage devices with the self-clearing system 100 provided as a smart catheter.

Overall, our experimental results demonstrate a novel non-pharmaceutical approach of using magnetic microactuator-enabled smart catheters 100 to treat IVH in situ using externally applied magnetic fields. It was demonstrated that these magnetic microactuators 102 may expedite the removal of blood from the ventricle, maintain a lower ventricle volume, and increase the survival rate of IVH-suffering animals. These types of magnetic microactuator-embedded smart catheters 100 may improve the lifetime and the reliability of chronically implanted catheters that are critical in a number of neurological, urological, cardiovascular, and other drug delivery applications.

In this experiment, the microactuators 102 were fabricated in a standard cleanroom environment. Starting from a 100 mm p-type single-side polished silicon wafer commercially available from SILICON QUEST™, 50 nm of silicon dioxide was deposited by plasma-enhanced chemical vapor deposition, via a system commercially available from AXIC™, to function as a release layer. Next, a layer of polyimide PI-2525, commercially available from HD MICROSYSTEM®, was spun coated at 1750 rpm and cured in a nitrogen oven up to 350° C. to create a final thickness of 11 μm. A 50-nm-thick Cr was evaporated using an AIRCO® E-Beam Evaporator as the etch mask 124 for the polymer layer. Cr mask was photo-patterned using AZ9260 and etched using a Cr etchant. The polyimide structural layer 108 was etched using O₂ plasma at 20 sccm, 50 mTorr and 150 W RF power using an Advanced Oxide Etcher commercially available from SURFACE TECHNOLOGY SYSTEM™. Cr mask was then removed using Cr-16.

Next, 100 nm of Au was sputtered globally as a conduction layer 110 and 200-μm-thick electroplating mold was photo-patterned using a negative photoresist, such as a BPN-65A commercially available from ROHM HAAS™. The nickel ferromagnetic elements were in a 2L plating solution maintained at 60° C. with a 40-mA direct current for four hours. The plating solution contained 1 M nickel sulfamate, 0.4 M boric acid, and 10 g sodium dodecyl sulfate. The electroplated elements varied from 80 to 130 μm in thickness depending on the actuator geometry. Afterward, the exposed Au conduction layer 110 was stripped using a wet etchant. The microactuators 102 were released from the wafer using a buffered oxide etchant. To create the self-clearing catheter 100, the released sample was rolled and inserted into a catheter lumen 104 such as Model G0664 commercially available from COOK MEDICAL™.

As shown in Table 1 below, provided as a non-limiting example, the self-clearing system 100 may be designed with the following dimensions:

TABLE 1

| Type | Straight | Serpentine |
|---|---|---|
| Beam length [μm] | 600 | 3000 |
| Beam width [μm] | 75 | 55 |
| Beam thickness [μm] | 11 | 11 |
| Tip volume [×10⁻³ mm³] | 46 | 40 |
| Ni thickness [μm] | 130 | 80 |
| Ni long axis [μm] | 775 | 905 |
| Aspect ratio | 5.9 | 11.3 |

COMSOL Multiphysics V5.0, commercially available from COMSOL®, was used for the finite element analysis of device deflection and stress distribution. Each device design of the self-clearing system 100 was configured to have the material property of polyimide with a density of 1300 kg/m³, Young's modulus of 2.45 GPa, and a poison ratio of 0.3. Using the solid mechanic's module, each flexure 112, 114 was fixed on one end and a vertical point load ranging from 1 to 100 μN was applied to the free end to evaluate the static deflection and the stress distribution. Lagrange strain was used to account for the large flexure deformation.

To characterize the static response, each sample was positioned along the long axis of a bespoke solenoid electromagnet (cylindrical permalloy core, 1-in-diameter, and 6-in-tall with 300 turns) and down to the edge of a glass slide. The distance between the sample and the electromagnet surface was kept at 7 mm to minimize the magnetic flux density gradient (<0.1 T/m) while maintaining an adequate amount of parallel magnetic field strength. A DC power source, such as a PWS2326 commercially available from TEKTRONIX®, was used to supply the current to the electromagnet. The amount of magnetic flux density at the position of actuators was measured using a gaussmeter. During actuation, the electromagnet along with actuator positioning glass slides was placed horizontally and the amount of deflection angle was optically measured using a digital SLR camera, such as a CANON® 50D. The magnetic flux density was varied from 0 to 22 mT for the straight beamed device and from 0 to 17 mT for the serpentine device.

To quantify the dynamic response, each sample was mounted on a glass slide and placed in a beaker filled with DI water. The device was then actuated using a bespoke iron-core electromagnet that was driven using a RIGOL® DG1022 signal generator coupled with an AE TECHRON® 7224 DC enabled AC amplifier between 1 and 100 Hz at 15 mT. The dynamic motion of the microactuator 102 at each frequency interval was captured using a Chronos 1.4 high-speed camera commercially available from KRON TECHNOLOGIES™. The amplitude of the actuator was then quantified using image tracking.

A 50 ml glass bottle was used to mimic the ventricular chamber. The bottle was sealed using a screw cap with two through holes allowing placement of the inlet and outlet tubing. A piece of a central venous catheter, such as Model G0664 commercially available from COOK MEDICAL® was attached to the outlet inside the glass bottle chamber. The through-holes on the screw cap were sealed using a silicone adhesive to fix the tubings in place. For the catheters used for flushing, a three-way stopcock was introduced to the outlet of the ventricle chamber. A 10 mL syringe with 2 mL of 1×PBS was attached to one of the inlets of the valve. A variable speed peristaltic pump was used to drive fluid into the chamber at 1.4 ml/min, about twice the rate of average cerebral spinal fluid production in humans. Two pressure sensors, such as a PRESS-S-00 commercially available from PENDOTECH® were connected to the inlet and the outlet tubes.

A mixture of porcine blood and 1×PBS at 50:50 vol was used to mimic hemorrhagic CSF. The fresh blood of euthanized pigs was mixed with 10 USP units of heparin/ml and stored in a refrigerator at 4° C. for 1 week prior to the experiment. At the time of testing, protamine sulfate was added (10 mg per 100 USP heparin) to reverse the effects of anti-coagulant and to facilitate blood clot formation. The rheological behavior of our blood mixture was studied using a cone and plate rheometer. Steady shear tests were performed at 37° C. Briefly, we used a controlled stress rheometer, such as the ARG2 commercially available from TA INSTRUMENTS®, with a 20-mm-diameter cone with a 1° angle attachment and 0.040 mL of the blood mixture for testing steady shear rates ranging from 0.1 to 500 s^{-1.} The results demonstrate the established shear thinning behavior of blood. The typical viscosity of blood ranges from 3.5 to 5.5 cP depending on the hemodynamic conditions. At the highest shear rate (500 s⁻1) the apparent viscosity of the blood mixture was 12.33 cP, which is a higher-than-normal range found in humans. Since conventional catheter occlusion is unpredictable with a wide range of time to failure, a layer of fibrin matrix gel was applied on the catheter surface surrounding the inlet pore to further promote blood clots attachment. The fibrin gel was made from a mixture of fibrinogen (38 mg/ml) and thrombin (37 mg/ml).

Each device was subjected to four hours of circulation in the circulating setup during which the differential pressure was recorded continuously (12 samples/min). For Control (n=4) and Flush (n=6) groups, single-pore catheters without any microactuators were tested. For Treatment groups, self-clearing catheters with either straight (n=5) or serpentine flexure designs were tested (n=6). During each experiment, the time-varying magnetic field was applied using a permanent magnet (McMaster-Carr, Elmhurst, IL, USA) affixed to a DC motor 120 from 20 mm away spinning at 8 Hz. Using the differential pressure recording, the time to reach a threshold pressure (i.e., time to occlusion, TTO) and the time over a threshold pressure (TOT, 40 mmHg) was calculated. The TTO and TOT for different conditions were compared using one-way ANOVA with Tukey's HSD post-hoc analyses with p<0.05 as statistical significance.

The ultrastructure of hematoma fragments was analyzed for the integrity of the fibrin fiber network. All blood-PBS solution exiting the VC was collected directly into a 200 mL 4% glutaraldehyde and 4% paraformaldehyde fixative for preparation for SEM hematoma fragment analysis. The blood-PBS fixed solution was prepared for SEM analysis. Samples were washed with three cycles of PBS (five minutes each). Secondary fixation was performed with 1% osmium tetroxide for one hour. The fragments were rinsed in de-ionized water three times at five minutes per cycle. All samples were dehydrated in a series of ethanol solutions of increasing concentration for ten minutes each (10%, 30%, 50%, 70%, 85%, two times at 95%, and three times at 100%) followed by critical point drying. Finally, samples were placed on aluminum mounts and carbon-coated prior to SEM. A grading scale was developed to semi-quantitatively measure the samples. Samples were analyzed and scored by a board-certified (American College of Veterinary Pathologists) clinical pathologist. All control (n=36) and treatment (n=18) samples were analyzed and scored by a board-certified (American College of Veterinary Pathologists) clinical pathologist.

For the in vivo evaluation, thirteen cross-bred domestic swine weighing 25.0-31.6 kg were used. The time-to-occlusion results were used to estimate the sample size of n=3 with the power of 0.8 and a of 0.05. This number was doubled for a target of n=6 to ensure that there was enough power to account for potential variations in animal testing. A post hoc analysis was also performed using the hazard ratio of 0.08, as shown below in Table 2. Using the hazard ratio, the acceptable sample size of n=4 per group was calculated with the following parameters: β=0.8, α=0.05, median survival time=7 d, and the planned average length follow-up=42 d.

TABLE 2

| Characteristic | HR¹ | 95% CI¹ | p-value |
|---|---|---|---|
| Group | 0.08 | 0.01, 0.68 | 0.0221 |

¹HR = Hazard Ratio, CI = Confidence Interval

Each pig was pre-medicated with 0.2 mg/kg midazolam, commercially available from HIKMA PHARMACEUTICALS™, and 0.03 mg/kg dexmedetomidine, commercially available from ZOETIS®, and induced with isoflurane delivered by face mask until tracheal intubation was possible. Anesthesia was continued with isoflurane delivered in 100% oxygen. Mechanical ventilation, pulse oximetry, capnography, indirect blood pressure measurement, temperature measurement, left jugular central line placement, and intravenous fluid therapy (10 mL/kg/h) were performed. Analgesia was provided by the pre-operative transdermal application of 2.7 mg/kg fentanyl solution (4-d expected activity). Intramuscular injection of 5 mg/kg ceftiofur (7-d expected activity) was used for antibiotic treatment.

To induce IVH, a right side subtemporal craniectomy was performed using a standard aseptic technique. To measure intraventricular ICP, a 1.25-in. 18-gauge intravenous cannula was directed through the intact dura mater into the right lateral ventricle. The correct ventricular location was confirmed by drainage of CSF. Non-compliant tubing (pre-filled with saline) was connected tightly and attached to a transducer and monitor. To inject the blood-thrombin mixture, a second 1.25-inch 18-gauge intravenous cannula was then placed into the lateral ventricle, more rostrally. The drainage of CSF again confirmed accurate placement. A total of 10 ml of autologous blood and 140 units of thrombin was injected through this cannula as determined using preliminary studies. Blood was drawn from the jugular catheter using the sterile technique in three equal aliquots. Each aliquot was briefly agitated with thrombin in a syringe and then injected. Each time ICP rose above 50 mmHg, the injection was paused until ICP was<50 mmHg. This second cannula was then withdrawn.

The puncture hole in the dura mater was slightly enlarged with a #11 scalpel blade, and the ventricular catheter was inserted through the tract that had been used for blood injection. In six pigs, a Control VP shunt was placed. The ventricular catheter was custom-made for this study using a modified central venous access device, with only one single inlet pore. In seven pigs, a Treatment shunt was placed. The custom-made ventricular catheter had a microactuator placed at the single inlet pore. The correct ventricular location was confirmed by rapid drainage of sanguineous CSF. ICP recording was continued until thirty minutes after the placement of the ventricular catheter.

The ventricular catheter was anchored to the skull by a pre-placed polydioxanone suture through the periosteum and loose fascia. It was connected to a pre-placed low-pressure valve and distal catheter which had been filled with saline, creating a VP shunt. The valve was anchored to the periosteal tissue of the posterior skull by a suture that encircled the connection of the ventricular catheter to the valve. The valve was pumped, and the functionality of the VP shunt was confirmed by sanguineous CSF entering the valve and saline escaping from the distal catheter. The distal catheter was placed in the peritoneal cavity and the paracostal incision closed. The fascia of the temporalis muscle was closed with a simple continuous polydioxanone suture. The skin was closed with simple continuous subcuticular and intradermal poliglecaprone sutures, followed by the application of skin glue.

CT was performed prior to and immediately following surgery (day 0). In the Treatment group, actuation was performed immediately after post-operative CT for thirty minutes using a bespoke electromagnet that was powered using a RIGOL™ DG1022 signal generator coupled with an AE TECHRON® 7224 DC enabled AC amplifier. The electromagnet has a maximum magnetic field strength of 50 kA/m which is orders of magnitude below the necessary magnetic field strength for transcranial magnetic stimulation. Pigs were then recovered from anesthesia. Repeat CT was performed at weeks 1, 3, and 5. Pigs were again premedicated with 0.2 mg/kg midazolam and 0.03 mg/kg dexmedetomidine, then had oxygen and isoflurane delivered by face mask throughout the CT. In the Treatment group, repeat actuation was performed immediately after each CT. The first CT was five to seven days post-operatively so that the first actuation was performed on day five or six in the Treatment group.

The size of the ventricles was measured using a DICOM viewer, such as the OSIRIX LITE® 10.0.1 commercially available from Pixmeo SARL Bernex. The ventricle (i.e., region of interest) from each CT scan was outlined manually by three measurers. The volume of the outlined region of interest was calculated by the DICOM viewer and compared. One-way ANOVA with Tukey's HSD post hoc analysis showed no statistical significance between the three measurers (p=0.095) using RStudio. To compare the difference in ventricle volumes in the Treatment vs. the Control group, two-way ANOVA was performed to see the effects of time and the type of catheters using RStudio. Post hoc pairwise comparisons were made using Tukey's HSD test.

Kaplan-Meier analysis of shunt survival was used to compare Treatment and Control groups using RStudio. Obstruction of the shunt by hematoma was considered an event. Both for fatal and non-fatal shunt obstructions causing a neurological decline, it was noted the increased size of the lateral ventricles on CT, and we verified the shunt obstruction by hematoma at necropsy when these animals were sacrificed at day 42. Death of the pig due to infection was censored.

Advantageously, the self-clearing system 100 may rapidly remove hematoma while also militating against infection, off-target tissue damage, and other complications including ventriculitis. Desirably, the self-clearing system 100 may also be implantable and externally controlled.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

What is claimed is:

1. A self-clearing system comprising:
a microactuator including a polyimide structural layer and a conduction layer, the polyimide structural layer having a main body and a flexure, the flexure coupled to the main body at a first terminal end of the flexure, the conduction layer is coupled to the polyimide structural layer;
a catheter lumen coupled to the microactuator, the catheter lumen is configured to accept the microactuator; and
an actuation device configured to wirelessly engage the microactuator.

2. The self-clearing system of claim 1, wherein the microactuator is coupled to the catheter lumen by a second terminal end of the flexure.

3. The self-clearing system of claim 2, wherein the flexure is substantially pliable.

4. The self-clearing system of claim 3, wherein the flexure has a rectangular cross-sectional shape.

5. The self-clearing system of claim 3, wherein the flexure has a curved cross-sectional shape.

6. The self-clearing system of claim 5, wherein the flexure has a serpentine cross-sectional shape.

7. The self-clearing system of claim 1, wherein the conduction layer includes a gold material.

8. The self-clearing system of claim 1, wherein the conduction layer is coupled to the polyimide structural layer by electroplating with a solution that includes at least one of nickel sulfamate, boric acid, and/or sodium dodecyl sulfate.

9. The self-clearing system of claim 1, wherein the actuation device includes a magnet.

10. The self-clearing system of claim 9, wherein the actuation device further includes a motor coupled to the magnet, wherein the motor is configured to selectively rotate the magnet.

11. A method of manufacturing a microactuator system configured to remove a blood clot, the method comprising the steps of:
providing a base layer, a release layer, a polyimide structural layer, and a conduction layer;
disposing a release layer on the base layer;
disposing a polyimide structural layer on a release layer;
etching the polyimide structural layer; and
coupling a conduction layer on the polyimide structural layer, thus forming a microactuator.

12. The method of claim 11, wherein the step of etching the polyimide structural layer includes disposing an etch mask on the polyimide structural layer and photo-patterning the etch mask.

13. The method of claim 12, wherein the etch mask is removed after the step of etching the polyimide structural layer.

14. The method of claim 11, wherein the step of coupling the conduction layer to the polyimide structural layer includes electroplating the conduction layer.

15. The method of claim 11, wherein the step of disposing a polyimide structural layer on a release layer includes spin coating the polyimide structural layer.

16. The method of claim 11, wherein the step of etching the polyimide structural layer includes etching the polyimide structural layer with a chromium etchant.

17. A method of using a self-clearing system configured to remove a blood clot, the method comprising the steps of:
providing a microactuator including a polyimide structural layer and a conduction layer, the polyimide structural layer having a main body and a flexure, the flexure coupled to the main body at a first terminal end of the flexure, the conduction layer is coupled to the polyimide structural layer;
disposing the microactuator within a catheter lumen;
coupling a second terminal end of the flexure to the catheter lumen; and
engaging the microactuator wirelessly with an actuation device.

18. The method of claim 17, wherein the actuation device includes a magnet which wirelessly engages the microactuator with a magnetic force.

19. The method of claim 18, wherein the actuation device further includes a motor which rotates the magnet to intermittently engage the microactuator.

20. The method of claim 19, wherein the actuation device further includes a controller which adjusts at least one of a rate and a duration of the engagement between the magnet and the microactuator.
